(19) United States
(12) Patent Application Publication
(10) Pub. No.: US 2010/0331276 A1
(43) Pub. Date: Dec. 30, 2010

(54) TOPICAL COMPOSITIONS AND THE USE THEREOF

(75) Inventor: Hugues Gatto, Saint-Paul (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: VIRBAC SA, Carros Cedex (FR)

(21) Appl. No.: 12/881,042

(22) Filed: Sep. 13, 2010

Related U.S. Application Data
(62) Division of application No. 11/910,684, filed on Oct. 4, 2007.

(30) Foreign Application Priority Data
Apr. 4, 2005 (FR) 0503291

(51) Int. Cl. A61K 31/702; A61K 31/7004; A61P 17/00

(52) U.S. Cl. 514/54; 514/23

(57) ABSTRACT

The invention relates to a composition for topical application as well as the use of such a composition. The invention is characterised in that it comprises at least one first and one second mono- or oligosaccharide, each of said first and second mono- or oligosaccharides being capable of limiting the adhesion of microorganisms on the skin of warm-blooded animals with coats.

This application is a divisional of U.S. application Ser. No. 11/910,684 filed Oct. 4, 2007, which is a U.S. national stage of PCT/FR2006/000730 filed Apr. 4, 2006, whose contents are hereby incorporated by reference herein in their entirety.

### FIELD OF THE INVENTION

This invention relates to compositions for topical applications, which compositions are suitable for limiting the adhesion of microorganisms on the skin of warm-blooded animals with coats. It relates in addition to the uses of such compositions in the veterinary field.

### BACKGROUND OF THE INVENTION

Animal skin is constantly attacked by pathogenic microorganisms. The horny layer of epidermis, due to its pH, its relatively low water content and the presence of antibiotic peptides having a bactericidal action, acts as the first barrier against these pathogenic microorganisms.

Nevertheless, changes in the structure of the epidermis, such as increased moisture or skin wounds, promote the colonisation and infection of the skin by the pathogenic microorganisms.

Antibiotics make it possible to effectively combat these pathogenic microorganisms, owing to their bacteriostatic and/or bactericidal actions.

Nonetheless, in consideration of the widespread, and sometimes excessive, use of antibiotics, the bacteria have developed a resistance to antibiotics limiting and even cancelling their antibacterial effects.

This is one of the reasons for which we seek to develop new means for combating pathogenic microorganisms.

It is known that the surface of some bacteria comprise lectins, which enable them to recognise and bind to sugar radicals specific to the glycoproteins and glycolipids located at the surface of the epithelial cells. It has been demonstrated in vitro that by miming the sugar radicals of the glycoproteins and glycolipids of epithelial cells, certain saccharides limit the adhesion of bacteria such as Pseudomonas aeruginosa to glycoproteins such as fibronectins (“Inhibition of Pseudomonas aeruginosa adhesion to fibronectin by PA-IL and monosaccharides: involvement of a lectin-like process, Julie Rebiere-Huët, Patrick Di Martino, and Christian Hulen. Can. J. Microbiol./Rev. Can. Microbiol. 50(5): 303-312 (2004)).

These so-called “anti-adhesive” properties of certain saccharides have been used in humans, for example in deodorants, as described in particular in the documents published under numbers U.S. Pat. No. 4,518 517 and EP A1 0 561 489.

However, the skin that covers the body of warm-blooded animals with coats is different from that of humans by its lower thickness, by the abundance of hair that constitute the coat and by the development of sebaceous glands, while there are very few sudoriferous glands. It is therefore not possible to extrapolate the relationships of interactions between bacteria (and more generally pathogens) and human skin to those existing between bacteria and the skin of warm-blooded animals with coats.

### SUMMARY OF THE INVENTION

In consideration of the above, a problem that the invention is intended to solve is that of developing a composition for topical application suitable for preventing and/or reducing the presence of microorganisms, and in particular pathogens, on the skin of warm-blooded animals with coats.

The solution to this problem, as proposed by the invention, first involves a composition for topical application, characterised in that it comprises at least one first and one second mono- or oligosaccharide, each of said first and second mono- or oligosaccharides being capable of limiting the adhesion of microorganisms on the skin of warm-blooded animals with coats.

The pathogenic microorganisms considered in particular are Staphylococcus intermedius, Pseudomonas aeruginosa and Malassezia pachydermatis. In particular, warm-blooded animals with coats are companion animals and especially dogs, cats and horses.

The invention also involves the use of a first and a second mono- or oligosaccharide, wherein each of said first and second mono- or oligosaccharides are capable of limiting the adhesion of microorganisms, for the preparation of a topical composition intended to prevent, to help to control or to treat skin conditions of warm-blooded animals with coats.

In addition, it involves a composition including charged micro- or nanoparticle carriers or non-charged micro- or nanoparticle carriers, which carriers include at least one active substance such as a mono- or oligosaccharide.

The presence of a single mono- or oligosaccharide capable of limiting the adhesion of microorganisms in a composition makes it possible to specifically inhibit the adhesion of a limited number of families of specific pathogenic microorganisms. However, when, according to the invention, a composition includes at least two mono- or oligosaccharides, each of said mono- or oligosaccharides being capable to limit the adhesion of microorganisms, the inhibition of the microorganisms affects a much larger number of families and an effective inhibition of the microorganisms is observed, so that the adherence of the pathogenic microorganisms on the skin of warm-blooded animals with coats is considerably reduced. The addition of a third mono- or oligosaccharide is also highly advantageous. Nevertheless, the addition of numerous other mono- or oligosaccharides appears to be of only minor benefit.

### MODE(S) FOR CARRYING OUT THE INVENTION

The invention can be better understood on reading the following non-limiting description below.

The composition according to the invention includes at least one first and one second mono- or oligosaccharide, which first mono- or oligosaccharide is different from said second mono- or oligosaccharide.

Monosaccharides are molecules consisting of three chemical elements: carbon, hydrogen and oxygen. They comprise 3 to 8 carbon atoms. Among the most common monosaccharides are the pentoses, such as arabinose, ribose, ribulose, xylose, xylulose and lyxose, and the hexoses, such as allose, altrose, fructose, galactose, glucose, gulose, idose, mannose, rhamnose, sorbose, talose and tagatose.

Oligosaccharides are homogeneous or heterogeneous oligomers constituted by the aforementioned monosaccharides. In practices, they are di-, tri- or tetramers of monosaccharides.

According to the invention, the first and second mono- or oligosaccharides are each capable of preventing and/or limiting the adhesion of microorganisms to the skin of warm-blooded animals with coats, in particular on the keratinocytes of the skin of these animals, and, more specifically, the corneocytes of the skin of these animals. They thus mime the sugar radicals of the glycoproteins and glycolipids, compete with these glycoproteins and glycolipids, and thus prevent the attachment of bacteria and yeast. Thus, by targeting the specific attachment mechanisms of pathogenic microorganisms to skin cells and tissue, the invention makes it possible to prevent or limit the process of colonisation and infection of pathogenic microorganisms on warm-blooded animals with coats.

Among the first or second monosaccharides capable of being used in the compositions of the invention, pentoses and hexoses are advantageously chosen. As a non-limiting example of pentoses, it is possible to cite D-arabinose. As non-limiting examples of hexoses, it is possible to cite D-fucose, L-fucose, D-galactose, D-glucose, D-mannose and L-rhamnose.

Among the first or second oligosaccharides capable of being used in the compositions according to the invention, homogeneous or heterogeneous oligomers constituted by the aforementioned monosaccharides will advantageously be chosen.

The aforementioned mono- or oligosaccharides all have at least anti-adhesive properties.

The composition according to the invention advantageously also includes a third mono- or oligosaccharide, which mono- or oligosaccharide is different from said first and second mono- or oligosaccharides.

This third mono- or oligosaccharide is advantageously itself capable of limiting the adhesion of microorganisms on the skin of warm-blooded animals with coats.

When the third mono- or oligosaccharide according to the invention is a monosaccharide, it is advantageously chosen from the pentoses and the hexoses. As a non-limiting example of pentoses, it is possible to cite D-arabinose. As non-limiting examples of hexoses, it is possible to cite D-fucose, L-fucose, D-galactose, D-glucose, D-mannose and L-rhamnose.

Among the third oligosaccharides capable of being used in the compositions according to the invention, homogeneous or heterogeneous oligomers constituted by the aforementioned monosaccharides will advantageously be chosen.

The first, second and/or third mono- or oligosaccharides are advantageously capable of reducing the production of TNF-α by the keratinocytes of the skin of warm-blooded animals with coats. It should be noted that this action on the production of TNF-α has recently been demonstrated, in vitro, on canine keratinocytes (“In vitro assays for canine keratinocyte activation modulation by fucose, arabinose and rhamnose”, C. Ibisch, P. Bourdeau, C. Cadiot and H. Gatto. 17^{th} ESVD-ECVD Congress, Copenhagen 2001). The reduction in the production of TNF-α has an immunomodulating effect and leads to a reduction in skin inflammation. It thus has an anti-irritating effect.

Advantageously, the first mono- or oligosaccharide of the composition according to the invention is L-rhamnose or a homogeneous oligomer constituted by L-rhamnose, the second mono- or oligosaccharide of the composition according to the invention is D-galactose or a homogeneous oligomer constituted by D-galactose and the third mono- or oligosaccharide of the composition according to the invention is D-mannose or a homogeneous oligomer constituted by D-mannose.

In addition, the composition according to the invention also advantageously comprises an alkylpolyglucoside. The alkylpolyglucosides (APG) according to the invention are substances constituted by one or more glucose residues and an alkyl group, resulting from the condensation of a fatty alcohol with glucose or one of its polymers.

As shown in example 2 below, the alkylpolyglucosides according to the invention are capable of preventing and/or limiting the adhesion of certain microorganisms, such as, in particular, Staphylococcus intermedius, on the skin of warm-blooded animals with coats. The presence of such alkylpolyglucosides in the compositions according to the invention therefore makes it possible to enlarge the adhesion-inhibiting action to a broader range of microorganisms less or not sensitive to the anti-adhesive properties of the mono- or oligosaccharides. Advantageously, the alkylpolyglucoside used in the composition according to the invention is lauryl diglucoside.

In addition, according to the invention, the first, second and/or third mono- or oligosaccharides and/or alkylpolyglucoside are advantageously contained in micro- or nanoparticle carriers. In practice, 5 to 90% of mono- or oligosaccharides and/or alkylpolyglucoside are contained in these carriers.

Among the micro- or nanoparticle carriers, it is possible to use all carrier systems enabling controlled release of the mono- or oligosaccharides according to the invention, such as microcapsules, microspheres, macromolecular complexes, nanospheres, nanocapsules, latex or vesicles.

As non-limiting examples of micro- or nanoparticle carriers, it is possible to cite the multilammellar vesicles sold by the NOVAVAX™ company under the name Novasomes™, spherical multilammellar vesicles with an onion structure called Spherulites™, microcapsules based on polyurethane, polyurea resin, polyamide resin, polyamide-polyurea resin, polycarbonate resin, polysulphonate resin and polysulphonamide resin.

In particular, the Spherulites™ can encapsulate hydrophilic or hydrophobic molecules with encapsulation efficiencies on the order of 90%. The molecules that they encapsulate are progressively released. The release of the encapsulated compounds can be controlled kinetically or thermodynamically.

The micro- or nanoparticle carriers according to the invention have a diameter between 0.01 μm and 150 μm. If these carriers are nanoparticle carriers, their diameter is preferably between 0.1 μm and 0.5 μm and, if they are microparticle carriers, their diameter is preferably between 1 μm and 50 μm.

In addition, the micro- or nanoparticle carriers according to the invention are charged or non-charged.

When these carriers are charged, they are advantageously cationic. In this case, owing to their positive charge, these carriers bind not only to the skin of animals, but also to the hair of their coats. They ensure a regular and controlled release of the mono- or oligosaccharides and/or alkylpolyglucoside at the surface of the skin and on the hair. Thus, even after a mechanical action, for example cleaning of the animal, the benefits of the composition according to the invention are preserved. This progressive release effect makes it possible to maintain the action of the mono- or oligosaccharides and/or alkylpolyglucoside on the adhesion of bacteria over time.

When the micro- or nanoparticle carriers according to the invention are not charged, they are called non-ionic. In this case, in addition to the aforementioned prolonged release effect, their presence promotes the diffusion of mono- or oligosaccharides and/or alkylpolyglucoside through the skin. Also, when the mono- or oligosaccharides and/or alkylpolyglucoside present in the carriers have an inhibitory effect on the activation of the keratinocytes and on the secretion of TNF-α, the skin inflammation reduction effects are improved. In addition, as the pathogenic microorganisms adhere more easily to inflamed skin than to healthy skin, the diffusion of mono- or oligosaccharides and/or alkylpolyglucoside, promoted by the presence of non-ionic micro- or nanoparticle carriers, through the skin, makes it possible to reduce skin inflammation and, de facto, indirectly limits the adhesion of bacteria, often itself the cause of the inflammation.

In an advantageous embodiment, the composition according to the invention comprises both cationic micro- or nanoparticle carriers and non-ionic micro- or nanoparticle carriers. In this case, the benefit lies in the respective contribution of each type of carrier to the reduction of the adhesion of the bacteria. First, the free mono- or oligosaccharides (not carried) and/or the free alkylpolyglucoside have a direct and immediate action on the adhesion of bacteria. Then the cationic micro- or nanoparticle carriers of the composition enable a direct and prolonged action of the mono- or oligosaccharides and/or alkylpolyglucoside on the adhesion of bacteria, while the non-ionic micro- or nanoparticle carriers exert an anti-irritant effect that diminishes skin inflammation and reduces the adhesion of bacteria.

Thus, the composition according to the invention has both an immunomodulating effect enhanced by a better diffusion of the mono- or oligosaccharides and/or alkylpolyglucosides encapsulated in non-ionic micro- or nanoparticle carriers through the skin, but also an anti-adhesive action prolonged by the presence of mono- or oligosaccharides and/or alkylpolyglucosides encapsulated in cationic micro- or nanoparticle carriers.

It should be noted that the composition according to the invention can also comprise antiseptic agents, chelating agents, keratolytic agents, keratoregulators, antiseborrheics and cleaning or softening agents.

As non-limiting examples of antiseptic agents capable of being used in the composition according to the invention, it is possible to cite chlorhexidine, hexachlorophene, parachlorometaxylenol, piroctone olamine and triclosan, taken alone or in a mixture. As non-limiting examples of chelating agents capable of being used in the composition according to the invention, it is possible to cite diethylene triamine pentaacetic acid and ethylenediaminetetracetate, taken alone or in a mixture. As non-limiting examples of keratolytic agents, keratoregulators or antiseborrheics capable of being used in the composition according to the invention, it is possible to cite lactic acid, salicylic acid, silver nitrate, sulphur and urea, ammonium lactate or zinc gluconate, taken alone or in a mixture. Finally, as non-limiting examples of cleaning or softening agents capable of being used in the composition according to the invention, it is possible to cite linoleic acid, cocoglucoside, decyl glucoside, disodium cocoamphodiacetate, disodium laureth, sodium dicusate, sodium stearate and sulphosuccinate, taken alone or in a mixture.

In practice, the topical compositions according to the invention are more specifically intended for treating the skin and mucous membranes and can exist in the form of ointments, creams, milks, pomades, wipes, syndets, solutions, gels, sprays, foams, suspensions, lotions, shampoos, or washing bases.

These compositions are used as cosmetic or pharmaceutical products for veterinary use. More specifically, they are used for hygiene of warm-blooded animals with coats, for helping to control, prevent or treat irritative dermatitis, atopic dermatitis, keratoseborrheic syndrome, external otitis, pyoderma or Malassezia dermatitis in warm-blooded animals with coats.

The invention therefore relates to the use of mono- or oligosaccharides in the preparation of a topical composition for helping to control or prepare a drug for the treatment of irritative dermatitis, atopic dermatitis, keratoseborrheic syndrome, external otitis, pyoderma or Malassezia dermatitis in warm-blooded animals with coats, as well as methods for treating these skin conditions in warm-blooded animals with coats, implementing the use of a composition containing mono- or oligosaccharides.

Of course, the choice of mono- or oligosaccharides and/or alkylpolyglucoside present in the composition, the number of mono- or oligosaccharides and/or alkylpolyglucoside present, and a possible encapsulation in carriers, is determined by the end use of the composition according to the invention.

In an example particularly suitable for helping to control atopic dermatitis in dogs, the composition according to the invention is a lotion comprising at least two specific monosaccharides, L-fucose and L-rhamnose, which monosaccharides are contained in Spherulite™ carriers. In another example, this time particularly suitable for external otitis in dogs, the composition according to the invention is a solution comprising three monosaccharides, L-rhamnose, D-mannose and D-galactose. In a final example, particularly suitable for the prevention of pyoderma in dogs, the composition is a shampoo comprising three monosaccharides, as described above, combined with an alkylpolyglucoside.

This invention will now be illustrated by means of examples 1 and 2 below:

### EXAMPLE 1

A study comparing the inhibition, by monosaccharides, of the adhesion of three different strains of Pseudomonas, P1, P2, P3, was conducted on canine corneocytes. The three monosaccharides studied are respectively D-galactose, D-mannose and L-rhamnose.

The three strains of Psedomonas were taken from dogs with infectious otitis. Corneocytes were taken from six different healthy dogs using D-squame™-brand adhesive pads, sold by the CuDerm™ Company.

Bacterial suspensions were produced, each containing one of the three strains P1, P2 or P3, and PBS (phosphate-buffered saline) with or without monosaccharides at two concentrations (0.05% and 0.1%). These suspensions were each placed on a corneocyte layer. These layers were then incubated in a humidity chamber. After incubation, the corneocytes were washed and stained. The adherent Pseudomonas were then quantified by computer-assisted image analysis.

The percentage of adherence of the strains in the presence of monosaccharides was then calculated with respect to the percentage of adherence of the strains without monosaccharides (positive control).

The adherence percentages were compared for the three strains P1 to P3 with respect to the positive control (100%).

The mean inhibition percentage for the three Pseudomonas strains was 25.6% for D-galactose, 19.4% for D-mannose and 30.8% for L-rhamnose.

It appears that each of the three monosaccharides limits the adhesion of Pseudomonas on the corneocytes. When the three monosaccharides are used in combination, a mean inhibition percentage of 53.4% is surprisingly observed.

Therefore, there is a conjugation of action when, according to the invention, a plurality of mono- or oligosaccharides, each having anti-adhesive properties, are present in the composition. This is at least the case when the composition according to the invention includes three mono- or oligosaccharides having these properties.

### EXAMPLE 2

A study comparing the inhibition by alkylpolyglucosides (Plantaren™ 1200) of the adhesion of three different strains of Staphylococcus intermedius, S1, S2 and S2, was conducted on canine corneocytes.

The three strains of Staphylococcus intermedius were taken from dogs with pyoderma (canine pyoderma). Corneocytes were taken from different healthy dogs, not having undergone a topical or systemic treatment in the three weeks before the sample was taken, using D-squame™-brand adhesive pads, sold by the CuDerm™ company.

Bacterial suspensions were produced, each containing one of the three strains S1, S2 or S3, and PBS (phosphate-buffered saline) with or without saccharides at a concentration of 1%. These suspensions were each placed on a corneocyte layer. These layers were then incubated in a humidity chamber. After incubation, the corneocytes were washed and stained. The adherent Staphylococcus intermedius were then quantified by computer-assisted image analysis.

The percentage of adherence of the strains in the presence of saccharides was then calculated with respect to the percentage of adherence of the strains without saccharides (positive control).

The adherence percentages were compared for the three strains S1 to S3 with respect to the positive control (100%).

The mean inhibition percentage for the three Staphylococcus intermedius strains was 47.71% for alkylpolyglucosides.

It thus appears that alkylpolyglucosides limit the adhesion of Staphylococcus intermedius on corneocytes.

1. A method for treating, preventing, or helping to control a skin condition in a dog, comprising administering to a dog in need thereof a topical composition comprising at least one first and one second mono- or oligosaccharide,
wherein each of said first and second mono- or oligosaccharides is capable of limiting the adhesion of microorganisms on the skin of a dog.

2. The method of claim 1, which is a method of limiting the adhesion of microorganisms on the skin of a dog, wherein the composition also comprises a third mono- or oligosaccharide, which third mono- or oligosaccharide is capable of limiting the adhesion of microorganisms.

3. The method of claim 1, wherein the mono- or oligosaccharides are each selected from the group consisting of D-arabinose, D-fucose, L-fucose, D-galactose, D-glucose, D-mannose and L-rhamnose and their homo- or heterogeneous oligomers.

4. The method of claim 1, wherein the first mono- or oligosaccharide is L-rhamnose, the second mono- or oligosaccharide is D-galactose and the third mono- or oligosaccharide is D-mannose, or their homogeneous oligomers.

5. The method of claim 1, which is a method of reducing the production of TNF-α by the keratinocytes of a dog, wherein the composition also comprises an alkylpolyglucoside capable of limiting the adhesion of microorganisms.

6. The method of claim 5, wherein the alkylpolyglucoside is lauryl diglucoside.

7. The method of claim 1, wherein the mono- or oligosaccharides and/or alkylpolyglucosides are contained in micro- or nanoparticle carriers.

8. The method of claim 7, wherein the micro- or nanoparticle carriers are charged.

9. The method of claim 7, wherein the micro- or nanoparticle carriers are non-ionic.

10. The method of claim 7, wherein 5 to 90% of the mono- or oligosaccharides and/or alkylpolyglucosides present in the composition are contained in micro- or nanoparticle carriers.

11. The method of claim 1, wherein the skin condition is selected from the group consisting of irritative dermatitis, atopic dermatitis, keratoseborrheic syndrome, external otitis, pyoderma and Malassezia dermatitis.

12. The method of claim 1,
wherein the first mono- or oligosaccharide is present in the composition in an amount sufficient to reduce the production of TNF-α by the keratinocytes of the skin of the dog, and
wherein each of said first and second mono- or oligosaccharides is present in the composition in an amount sufficient to limit the adhesion of microorganisms on the skin of the dog.

13. The method of claim 1, wherein the first mono- or oligosaccharide is L-rhamnose, D-fucose or L-fucose.

14. The method of claim 13, wherein the first mono- or oligosaccharide is L-rhamnose.

15. The method of claim 1, wherein the first mono- or oligosaccharide is capable of reducing the production of TNF-α by the keratinocytes of the skin of a dog.

16. The method of claim 1, wherein the composition is capable of reducing the production of TNF-a by the keratinocytes.

17. The method of claim 8, wherein the micro- or nanoparticle carriers are cationic.
